(19) United States
(12) Patent Application Publication
(10) Pub. No.: US 2016/0045201 A1
(43) Pub. Date: Feb. 18, 2016

(54) BLOOD FLOW DISRUPTION DEVICES AND METHODS FOR THE TREATMENT OF VASCULAR DEFECTS

(71) Applicant: Sequent Medical, Inc., Aliso Viejo, CA (US)

(72) Inventors: Robert Rosenbluth, Laguna Niguel, CA (US); Brian J. Cox, Laguna Niguel, CA (US)

(73) Assignee: Sequent Medical, Inc., Aliso Viejo, CA (US)

(21) Appl. No.: 14/926,423

(22) Filed: Oct. 29, 2015

Related U.S. Application Data
(63) Continuation of application No. 14/001,818, filed on Aug. 27, 2013.
(60) Provisional application No. 61/444,563, filed on Feb. 18, 2011.

(51) Int. Cl. A61B 17/12; A61F 2/90; A61F 2/962; A61F 2/07

(52) U.S. Cl. CPC A61B 17/12113; A61F 2/07; A61F 2/90; A61F 2/962; A61B 17/12118; A61B 17/12036; A61B 17/12172; A61F 2220/0058; A61F 2220/005; A61F 2210/0014; A61F 2250/0014; A61F 2002/077; A61F 2230/0006; A61F 2230/0065; A61B 2017/00867

(57) ABSTRACT

A blood flow disruption device for embolizing blood flowing into a vascular defect between a proximal vascular segment and a distal vascular segment, wherein the device includes a porous inner flow disruption element configured to extend through the defect between the proximal vascular segment and the distal vascular segment, whereby a first portion of the blood flowing into the inner flow disruption element from the proximal vascular segment is directed to flow into the defect and a second portion of the blood flowing into the inner flow disruption element is directed to flow into the distal vascular segment. A porous outer flow disruption element coaxially surrounds the inner flow disruption element and is radially expansible from a collapsed state to an expanded state. The outer flow disruption element, in its expanded state, promotes sufficient hemostasis of the first portion of the blood within the defect to embolize the defect.

### CROSS-REFERENCE TO RELATED APPLICATION

This application is a continuation application of U.S. application Ser. No. 14/001,818, filed on Aug. 27, 2013; which is a national phase filing, under 35 U.S.C. §371(c), of International Application Serial Number PCT/US2012/025390, filed on Feb. 16, 2012, which claims priority, under 35 U.S.C. §119(e), from U.S. Provisional Application No. 61/444,563, filed on Feb. 18, 2011. The disclosures of the aforesaid applications are incorporated herein by reference in their entireties.

### FEDERALLY SPONSORED RESEARCH OR DEVELOPMENT

Not Applicable.

### BACKGROUND

This disclosure relates to devices and methods for the treatment of vascular defects, particularly aneurysms. More specifically, it relates to devices and methods that provide embolization of defects such as vascular aneurysms.

The mammalian circulatory system includes a heart, which acts as a pump, and a system of blood vessels (the vascular system), which transports the blood throughout the body and back to the heart. Due to the pressure exerted by the flowing blood through the blood vessels, the blood vessels may develop a variety of vascular defects. One common vascular defect, known as an aneurysm, is characterized by an abnormal widening of the blood vessel. Typically, vascular aneurysms are formed as a result of the weakening of the wall of a blood vessel and the subsequent ballooning and expansion of the vessel wall. The rupturing of an aneurysm may have serious consequences. For example, should an aneurysm within a cerebral artery burst, the resulting cranial hemorrhaging could cause serious neurological damage, leading to disability or death.

Surgical techniques for the treatment of cerebral aneurysms typically involve a craniotomy requiring creation of an opening in the skull of the patient through which the surgeon can insert instruments to operate directly on the patient's brain. For some surgical approaches, the brain must be retracted to expose the parent blood vessel from which the aneurysm arises. Once access to the aneurysm is gained, the surgeon places a clip across the neck of the aneurysm, thereby preventing arterial blood from entering the aneurysm. Upon correct placement of the clip, the aneurysm will be obliterated in a matter of minutes. Surgical techniques may be effective treatment for many aneurysms. Unfortunately, surgical techniques for treating these conditions include major surgery procedures that often require extended periods of time under anesthesia involving high risk to the patient. Such procedures thus require that the patient be in generally good physical condition in order to be a candidate for such procedures.

Various alternative and less invasive procedures have been used to treat cerebral aneurysms without resorting to major surgery. Some such procedures involve the delivery of embolic or filling materials into an aneurysm. The delivery of such vaso-occlusion devices or materials may either fill the aneurysm directly, or they may promote hemostasis to fill the aneurysm cavity with an embolus (clotted blood). Vaso-occlusion devices may be placed within the vasculature of the human body, typically via a catheter, either to block the flow of blood through a vessel with an aneurysm through the formation of an embolus or to form such an embolus within an aneurysm stemming from the vessel. A variety of implantable, coil-type vaso-occlusion devices are known. The coils of such devices may themselves be formed into a secondary coil shape, or any of a variety of more complex secondary shapes. Vaso-occlusive coils are commonly used to treat cerebral aneurysms, but they suffer from several limitations, including poor packing density, compaction due to hydrodynamic pressure from blood flow, poor stability in wide-necked aneurysms and complexity and difficulty in their deployment, due to the frequent need for the deployment of multiple coils to treat an aneurysm.

Another approach to treating aneurysms without surgery involves the placement of sleeves or stents into the vessel and across the region where the aneurysm occurs. Such devices maintain blood flow through the vessel while reducing blood pressure applied to the interior of the aneurysm. Certain types of stents are expanded to the proper size by inflating within them a balloon catheter; these are referred to as balloon expandable stents. Other stents are designed to elastically expand in a self-expanding manner. Some stents are covered typically with a sleeve of polymeric material called a graft to form a stent-graft. Stents and stent-grafts are generally delivered to a preselected position adjacent a vascular defect through a delivery catheter. In the treatment of cerebral aneurysms, covered stents or stent-grafts have seen very limited use due to the likelihood of inadvertent occlusion of small perforator vessels that may be near the vascular defect being treated.

In addition, current uncovered stents are generally not sufficient as a stand-alone treatment. In order for a stent to fit through a microcatheter sized for use in small cerebral blood vessels, the density of the stent must typically be sufficiently small that when the stent is expanded, there is only a small amount of stent structure bridging the aneurysm neck. This small amount of stent structure may not block enough flow to cause clotting of the blood in the aneurysm. Consequently, uncovered stents are generally used in combination with vaso-occlusive devices, such as the coils discussed above, to achieve aneurysm occlusion.

The use of various aneurysm neck bridging devices or intraluminal flow diverters has been attempted. One limitation in their adoption and clinical usefulness is the time that it takes for the occlusion to take place. In most cases, the duration from implant to occlusion is several months. Further, it has been postulated that diverting only the inflow of blood to an aneurysm may subject the “dome” of the aneurysm to altered flow conditions that can, in some circumstances, cause a rupture and hemorrhage before the process of thrombosis is able to protect the dome.

What has been needed are devices, along with methods for their delivery and use in small and tortuous blood vessels, that can substantially block the flow of blood into an aneurysm, such as a cerebral and abdominal aneurysms, with a decreased risk of inadvertent aneurysm rupture or blood vessel wall damage. In addition, what has been needed are methods and devices suitable for blocking blood flow in cerebral aneurysms over an extended period of time without a significant risk of deformation, compaction or dislocation.

### SUMMARY

In one aspect, this disclosure describes a blood flow disruption device for embolizing blood flowing into a vascular defect between a proximal vascular segment and a distal vascular segment, wherein the device comprises a porous inner flow disruption element configured to extend through the defect between the proximal vascular segment and the distal vascular segment, whereby a first portion of the blood flowing into the inner flow disruption element from the proximal vascular segment is directed into the defect, and a second portion of the blood flowing into the inner flow disruption element is directed into the distal vascular segment; and an outer flow disruption element coaxially surrounding the inner flow disruption element and radially expansible from a collapsed state to an expanded state; wherein the outer flow disruption element, in its expanded state, creates sufficient hemostasis of the first portion of the blood within the defect to embolize the defect.

In the context of an arterial defect, a parent artery may have a vascular defect or aneurysm, and the non-defect or non-dilated portions or segments of the parent artery upstream and downstream from the defect may be referred to, respectively, as the upstream vascular segment and the downstream vascular segment. In the context of this disclosure, however, the non-dilated vascular segments on either side of the defect may more generally be referred to as the “proximal segment” and the “distal segment,” in relation to the deployment apparatus and method that are discussed below.

The disclosed embodiments facilitate the reconstruction of the vascular wall defect and promote the embolization of the defect external to the reconstruction. The disclosed embodiments also provide a high degree of flow disruption, and thus hemostasis, within the defect (e.g. aneurysm) that should be particularly beneficial in the case of a previously-ruptured vascular wall. Embodiments described herein are particularly useful for the treatment of vascular defects in the form of wide-necked and fusiform aneurysms, particularly wide-necked and fusiform cerebral aneurysms, aneurysms of the abdominal aorta, and similarly-shaped defects in other luminal organs.

In accordance with aspects of this disclosure, a blood flow disruption device includes an inner flow disruption element that forms a porous conduit configured for deployment intravascularly into a target vascular wall defect so as to span the defect, and at least one radially-expansible, porous outer flow disruption element coaxially surrounding a substantial portion of the length of the inner element, wherein the at least one outer element, when radially expanded within the target defect, forms a porous flow baffle that disrupts and slows the flow of blood through the defect, thereby promoting hemostasis within the defect. The hemostasis, in turn, results in embolization within the defect that significantly reduces the risk of further damage to the vascular wall at the defect, while promoting healing of the defect.

The inner and outer flow disruption elements may be combined to form a multi-element device, or the inner element and the outer element(s) may be deployed separately, in serial fashion. The device has a radially expanded state when deployed, and radially collapsed state that allows delivery through small catheters (e.g., microcatheters) to the target vascular site. Thus, the device may be delivered intravascularly through tortuous cerebral vasculature for deployment adjacent to or within an intracranial aneurysm.

In some embodiments, the inner element comprises a mesh, fabric, lattice, braid, weave, or fenestrated portion. In some embodiments, the inner element may be formed of a braid of filaments that may include monofilaments, wires, yarns or threads. The inner element may be substantially cylindrical in form. The outer flow disruption element(s) may also comprise a mesh, fabric, lattice, braid, weave, or fenestrated portion. Each outer element has at least one radially dilated or expansible portion having a diameter greater than the outer diameter of the inner element. In some embodiments, the outer element(s) may be formed of a braid of filaments. In some embodiments, the outer element(s) may have an undulating form.

In some embodiments, an outer flow disruption element may have at least one portion with substantially the same diameter or maximum transverse dimension of the inner flow disruption element. In some embodiment, both ends of the outer element substantially match the inner element in size. Because the inner element is generally sized to fit the parent vessel or vascular portion of the artery (although some over-sizing may be done to provide a good seal with the artery wall), the outer element may also have a portion that is sized to fit the parent vessel or vascular portion of the artery in the same manner.

The combination of the inner and outer flow disruption elements provides a synergistic effect in the treatment of aneurysms. Specifically, the inner element provides disruption of blood flow into the aneurysm sac and a matrix for healing and reconstruction of the parent artery lumen through the defect. Each of the outer elements provides flow disruption inside the aneurysm sac by forcing at least a portion of the flow within the sac to pass through multiple layers of flow-disruption material, thereby promoting thrombosis or embolization of the aneurysm. This large amount of flow disruption can facilitate sufficient flow stasis for significant embolization at the time of treatment or very soon thereafter. After embolization, the inner element provides a blood flow passage from the upstream vascular portion to the downstream vascular portion through the embolized defect, while the outer flow disruption element provides a structural matrix that supports and holds the embolism in place within the defect.

The inner element and each outer element may be attached to one another by means known in the art of attachment, including, but not limited to, mechanical connectors, welding, brazing, soldering, adhesives and the like. Alternatively, the elements may be left unconnected, and the outer element secured in position by the inner element.

The specific features and advantages of the device and methods disclosed herein will be more readily apparent from the detailed description that follows.

### BRIEF DESCRIPTION OF THE DRAWINGS

FIG. 1 illustrates a blood flow disruption device in accordance with a first specific embodiment of the disclosure, showing the device, partially in cross-section, installed within a vascular defect, before embolization of the defect has begun;

FIG. 2 is an elevational view of an inner flow disruption element of the device of FIG. 1, in accordance with an embodiment of the disclosure;

FIG. 3 is an elevational view of an outer flow disruption element of the device of FIG. 1, in accordance with an embodiment of the disclosure;

FIG. 4 illustrates a blood flow disruption device in accordance with second specific embodiment of the disclosure, showing the device installed within a vascular defect;

FIGS. 5-7 are longitudinal cross-sectional views of a blood flow disruption device in accordance with an embodiment of the disclosure, showing alternatives configuration for the outer flow disruption element;

FIG. 8 illustrates a flow disruption device in accordance with a third specific embodiment of the disclosure, showing the device installed within a vascular defect;

FIG. 9 is a diagrammatic cross-section view of an artery with an embolized vascular defect in accordance with the device embodiment of FIG. 8;

FIG. 10 illustrates a blood flow disruption device in accordance with a fourth specific embodiment of the disclosure, showing the device installed within a vascular defect;

FIG. 11 is a perspective view of an alternative configuration of an inner flow disruption element of a blood flow disruption device in accordance with an embodiment of the disclosure;

FIG. 12 is a detailed view of a portion of the inner flow disruption element of FIG. 8;

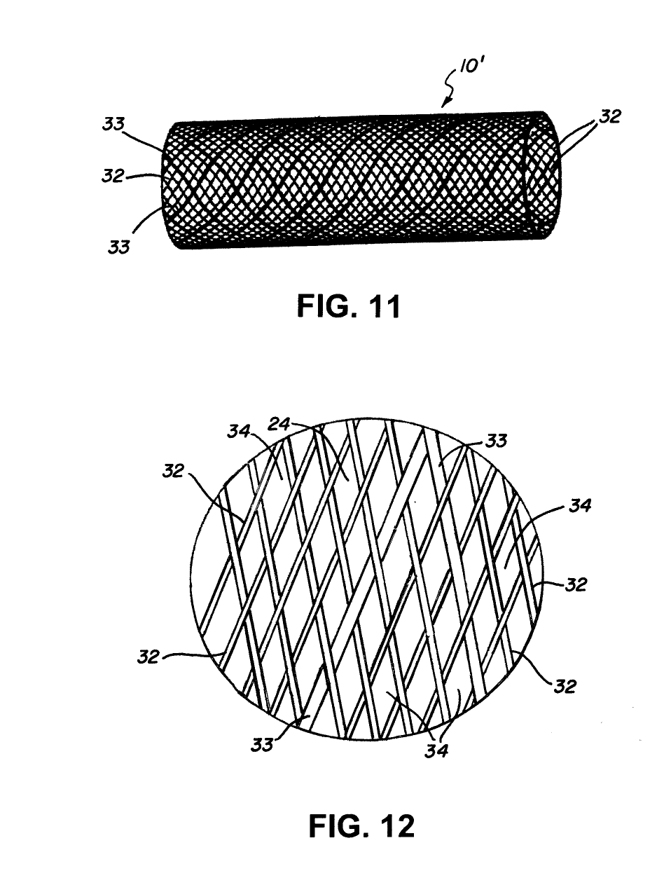

FIG. 11 is a semi-schematic view of a portion of an apparatus for forming the inner flow disruption element of a blood flow disruption device in accordance with an embodiment of the disclosure;

FIG. 13 is a semi-diagrammatic view showing sites on the human body from which deployment of a blood flow disruption device in accordance with the disclosure to an intracranial site of a vascular defect may be initiated;

FIG. 14 is a simplified view, partially in cross-section, of apparatus that may be used to deploy a blood flow disruption device in accordance with the disclosure;

FIGS. 15-18 are semi-diagrammatic views showing a method of deploying and installing a blood flow disruption device in accordance with an embodiment of the disclosure; and

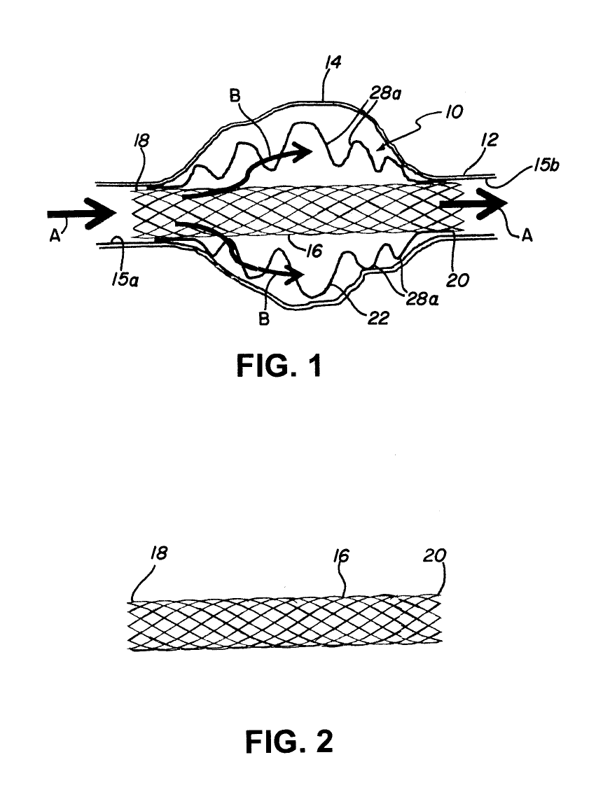

FIG. 19 is a view similar to that of FIG. 1, showing the installed blood flow disruption device within a vascular defect after completion of embolization of the defect.

### DETAILED DESCRIPTION

FIG. 1 illustrates a blood flow disruption device (“device”) 10 in accordance with an embodiment of the disclosure. The device 10 is shown installed in a blood vessel 12 (e.g., an artery) having a vascular wall defect 14 between an upstream or proximal vascular segment 15a and downstream or distal vascular segment 15b. As shown, the wall defect 14 is an aneurysm, in particular a fusiform aneurysm. The arrows A show the direction of blood flow through the blood vessel 12, while the arrows B show the disrupted blood flow, created by the device 10, through the distended portion of the blood vessel 12 in the area of the defect 14.

As shown, the device 10 comprises an inner flow disruption element 16 that has a radially expanded state in which it is configured as a porous tubular conduit. In the expanded state, the inner element 16 has an inflow end with a proximal fixation zone 18, and an outflow end with a distal fixation zone 20 (see FIG. 2). The fixation zones 18, 20 are dimensioned to engage the wall of the vessel 12 in the upstream vascular segment 15a and the downstream vascular segment 15b, respectively, so as to seat the device snugly within the vessel 12.

As mentioned above, the inner element 16 has a porous wall structure, as described below, so that a substantial portion of the blood flowing into its inflow end is directed radially out of the inner element 16 and into the vascular defect 14. The wall of the inner element 16 is, however, sufficiently solid to direct the remaining portion of the blood entering the inflow end into the distal vascular segment, whereby the inner element forms a reconstituted vascular lumen through the defect. To achieve the desired porosity, the inner element 16 is advantageously formed of a filamentous mesh, fabric, lattice, braid, or weave. Alternatively, the inner element 16 may be a fenestrated or perforated cylinder. For example, in some embodiments, the inner element 16 may be formed of a mesh or braid of filaments that may include polymeric monofilaments, metal wires, or fabric yarns or threads. In some embodiments, the filaments are highly elastic to provide a self-expanding characteristic. Exemplary materials include, but are not limited to, super-elastic nickel-titanium alloy (“nitinol”) and cobalt-chromium alloys.

The fixation zones 18, 20 may be provided with structure or materials that enhance the fixation of the inner element 16 within the vessel 12. Suitable biocompatible coatings and surface treatments that would enhance fixation are well-known in the art, as is the provision of surface microfeatures configured as hooks, barbs, dimples, or protrusions.

In some embodiments, the inner element 16 forms a substantially smooth inner surface with only the small undulations created by fenestrations and/or the interweaving of filaments. In some embodiments the inner element 16 may have circular, helical or longitudinal grooves, channels, ridges or concavities along a portion or substantially all of its inner surface. These grooves, channels, ridges or concavities may serve to encourage flow patterns that are beneficial to the maintenance of patency of the device and/or minimize inner surface thrombus formation that can pose a risk of embolic stroke if it dislodges and floats downstream. Exemplary constructions of vascular implants with a lumen having surface channels and the like are described in U.S. Pat. Nos. 6,776,194; 7,185,677; and 7,682,673, the disclosures of which are herein incorporated by reference in their entireties.

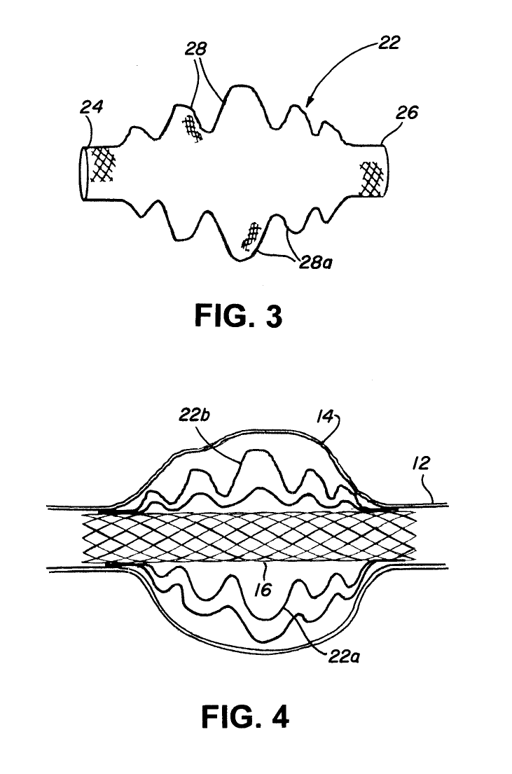

As shown in FIGS. 1 and 3, the device 10 further includes a radially expansible outer flow disruption element 22 coaxially surrounding at least a substantial portion of the inner element 16. The outer element 22 has an inflow end 24 and an outflow end 26 that may either be attached to the fixation zones 18, 20, respectively, of the inner element 16, or simply captured between the fixation zones 18, 20 and the wall of the vessel 12, as will be explained in more detail below. The outer element 22, like the inner element 16, is porous, and thus may advantageously be made of a filamentous material, such as a polymeric filament, metal wire, or fabric thread that is formed into a mesh, braid, fabric, or weave. Some embodiments may employ a first outer flow disruption element 22a and a second coaxial outer flow disruption element 22b, as shown in FIG. 4. Other embodiments may include more than two outer flow disruption elements arranged coaxially.

The outer element 22 has an expanded state, as shown, in which it has a measurably larger diameter than the inner element 16 (up to about five times the diameter of the inner element), and advantageously, a much larger surface area exposed to blood flow. In its expanded state, the outer element 22 forms a porous flow baffle within the vascular defect (aneurysm) 14 through which flows blood that has already flowed through the porous wall of the inner element 16. The baffling effect is achieved by the presence of wave-like undulations in the outer element 22 that form multiple layers that both increase the surface area exposed to blood flow, and further enhance the disruption, and thus slowing, of blood flow through the defect 14. Further, the undulations may provide mechanical support of the inner element 16, thus stabilizing the inner element and thus the entire device 10, thereby reducing the risk of movement and/or kinking of the inner element.

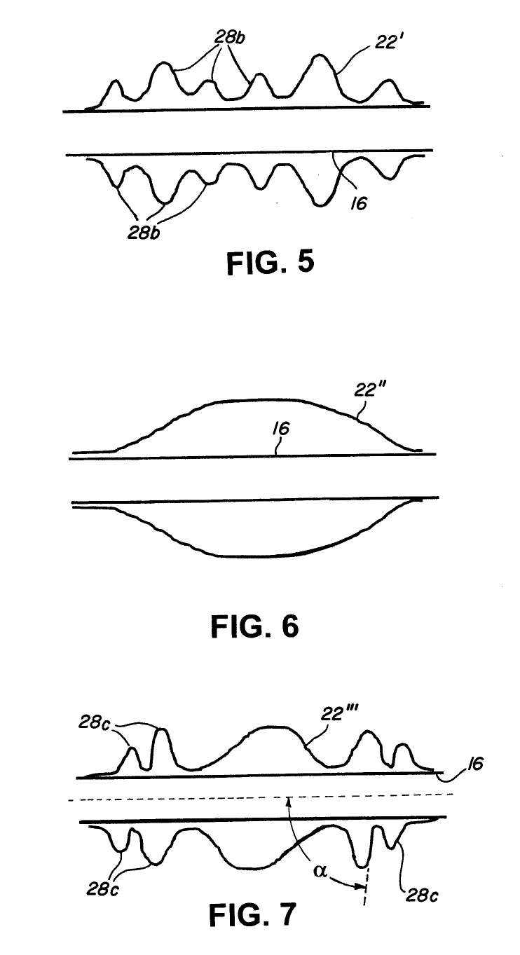

The undulations may assume a variety of forms. For example, FIGS. 1 and 3 show undulations 28a that are generally sinusoidal in form and that gradually increase in height (amplitude) from the ends of the outer element 22 toward its center. FIG. 5 shows an outer element 22′ having undulations 28b that vary in height arbitrarily. FIG. 6 shows an outer element 22″ that may be considered to lack undulations, consisting of a smooth, continuous, rounded or bulbous shape. FIG. 7 shows an outer element 22′″ having undulations 28c that are more densely spaced at the ends of the outer element than at the center. This arrangement of undulations 28c near the ends may reduce the risk of blood flow around the device 10 (“endoleaks”). As also shown in FIG. 7, the angle α defined between the outer element undulations and the longitudinal axis a of the inner element 16 may be between about 60 and 85 degrees, preferably between about 70 and 85 degrees, and more preferably between about 75 and 85 degrees. Each of these configurations for the outer element may be advantageous in particular situations or applications.

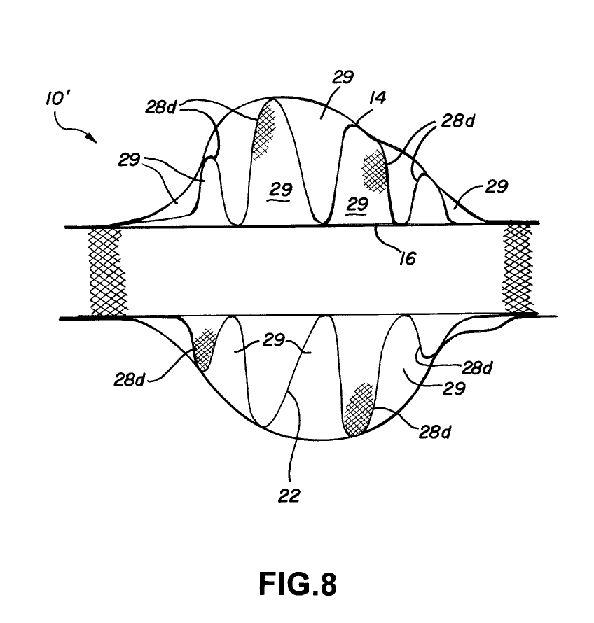

FIG. 8 illustrates a device 10′, in accordance with another embodiment, in which one or more undulations 28d of an outer element 22^{iv} contact, and are optionally attached to, the exterior surface of the inner element 16. In this configuration, the inner and outer elements define one or more closed spaces 29 surrounding the inner element 16. The number of such closed spaces 29 may be varied, and, in some embodiments, one or more of them may assume something resembling a toroidal configuration. It is understood that a torus is a surface of revolution generated by revolving a circle in three dimensional space about an axis coplanar with the circle. The cross-section of the closed spaces 29 in the device 10 will generally not have a circular cross-section, and may have a generally triangular or irregular shape. Thus, for the purposes of this disclosure, the term “toroidal configuration” shall include a surface of revolution generated by revolving a circular, triangular, or irregular shape in three dimensional space about an axis coplanar with the circular, triangular or irregular shape. In some embodiments, one or more substantially closed generally torodial spaces 29 may be created by deployment of porous mesh elements. In some embodiments, a plurality (advantageously, but not necessarily, between two and 12) of generally toroidal closed spaces 29 may be formed from porous mesh elements.

As illustrated in FIG. 8, the closed spaces 29 may be defined both between the inner element 16 and the outer element 22^{iv}, and between the outer element 22^{iv} and the vascular wall of the defect 14. In other embodiments, the outer flow disruption element may be configured so that closed spaces are formed only between the inner and outer elements, or only between the outer element and the vascular defect wall.

In the ensuing discussion, use of the reference numeral 22 in connection with the outer flow disruption element should be understood to include any or all of the above-described embodiments and variants 22′, 22″, 22′″, and/or 22^{iv}, as applicable.

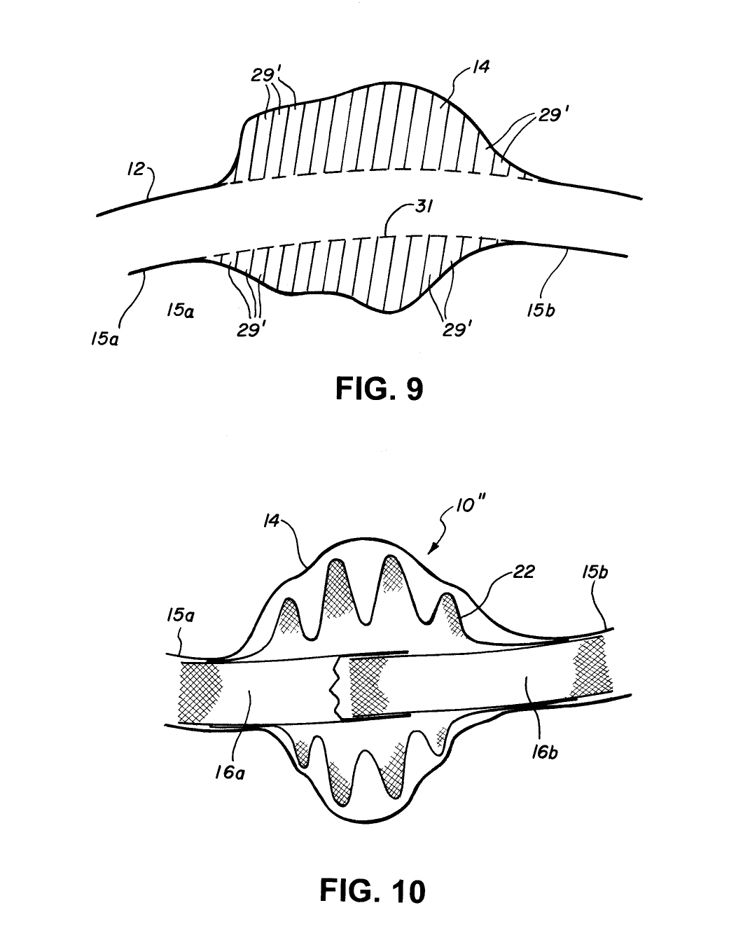

In some embodiments, as exemplified by the embodiment shown in FIG. 8 and discussed above, the outer element and the inner element may define one or more substantially closed spaces 29 that separate at least a portion of the vascular defect volume into a plurality of sub-volumes 29′ (FIG. 9) that occupy, in total, between about 40% and 100%, and advantageously between about 60% and 90%, of the total defect volume, where the total defect volume is the volume of the dilated segment (defect 14) of the artery that is outside of a virtual lumen 31 that is defined as an extension or continuation through the defect 14 of the undilated artery segments 15a, 15b, as shown in FIG. 9. Furthermore, in such embodiments, it is advantageous for at least one of the sub-volumes 29′ to be between about 10% and 80% of the total defect volume.

In some embodiments, at least some of the sub-volumes 29′ are filled with a biomaterial or devices as described herein. Optionally, the closed structures or sub-volumes may not be filled with a foreign body or material. Thus, they become filled with only blood upon implantation, and the body's own hemostasis and clotting mechanisms embolize the vascular defect volume. Accordingly, the devices and methods allow for a natural healing process to occur where the vascular defect may at least partially collapse or reduce in volume over time after treatment as the clotted blood organizes to form fibrous tissue. This can be advantageous compared to vascular defects that are substantially filled with devices, biomaterials or other foreign matter. Such devices, biomaterials or foreign matter can impinge on tissues or organs in a similar manner to an untreated aneurysm and thus cause undesired symptoms. Further, such devices, biomaterials or foreign matter can erode through the vascular defect wall into other tissue structures or organs over time, with potentially adverse consequences.

In some embodiments, the amplitude of the outer element undulations may be between about 63% and 300% of the diameter of the inner element 16. Thus, the diameter of the outer element 22 may, in some embodiments, range from about 225% to about 700% of the diameter of the inner element 16. The collapsed length of the outer element 22 may be between about 125% and 500% of the collapsed length of the inner element 16. In some embodiments, the outer element 22 defines a volume that is between about 125% and 500% the volume defined by the inner element 16.

The pore structure and large surface area of the outer element 22 provides sufficient flow disruption to promote rapid hemostasis of the defect (aneurysm) 14. In some embodiments, the device may provide sufficient flow disruption to substantially embolize the aneurysm such that when contrast agent is injected in a follow-up angiogram, no significant contrast can be seen outside the inner member within about 24 hours. The surface area of each of the inner element 16 and the outer element 22 may be between about 50 mm² and 10,000 mm². In some embodiments, the outer element 22 may have between about 1.25 times and 5.0 times the surface area of the inner element 16.

Optionally, the inner flow disruption element 16 and/or the outer flow disruption element 22 may be formed of filaments that may be reactive or responsive to either environmental changes or the input of energy. For example, the device 10 may respond to a temperature change using thermal shape memory as is known in the art of shape memory devices. Alternatively, the device 10 may react to energy delivered to the device 10 that causes it to increase in temperature. Thus, the device 10 may cause changes to the aneurysm wall or to blood contained within the device.

FIG. 10 illustrates a device 10″ in accordance with another embodiment, in which a first inner element segment 16a may extend from an upstream vascular segment 15a of the parent artery that is substantially non-dilated, or from an upstream part of the defect 14 to a point within the vascular defect. Subsequently, a second inner element segment 16b may extend from a first end placed within the downstream end of first inner element segment 16a in at least a partially over-lapping fashion, to a second end seated in the downstream vascular segment 15b. Thus, an inner flow disruption element formed from a plurality of inter-connected inner element segments may be used to reconstruct the parent artery and form a reconstituted lumen through the vascular defect.

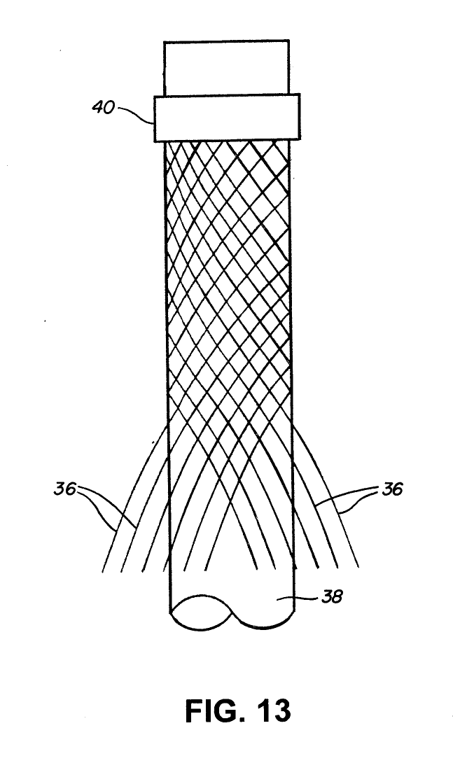

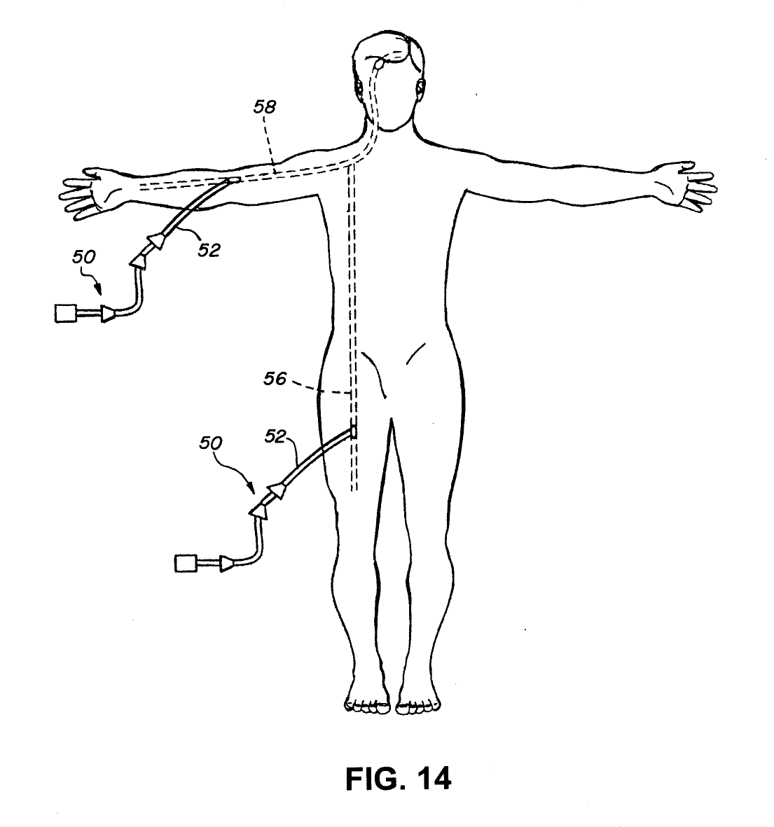

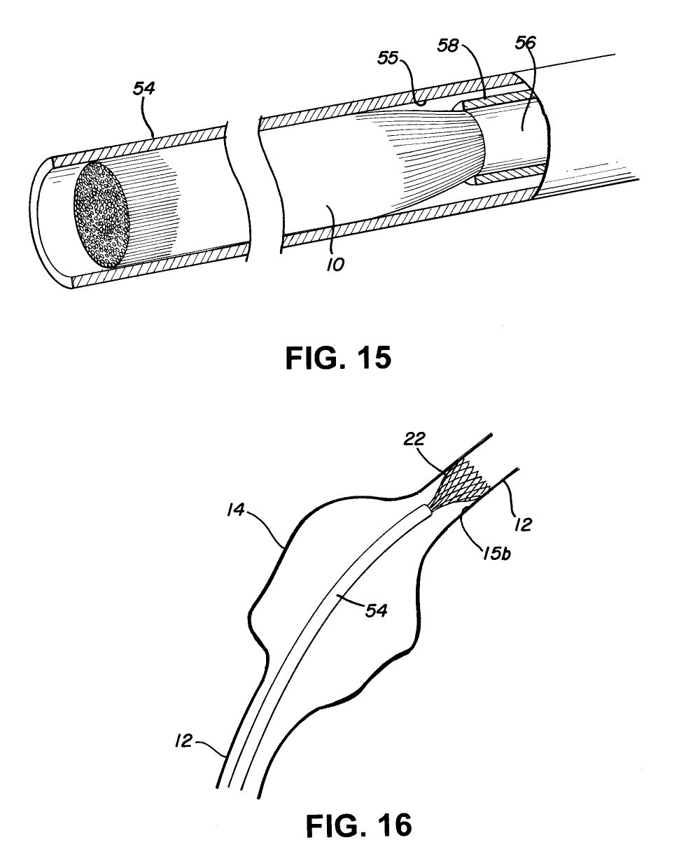

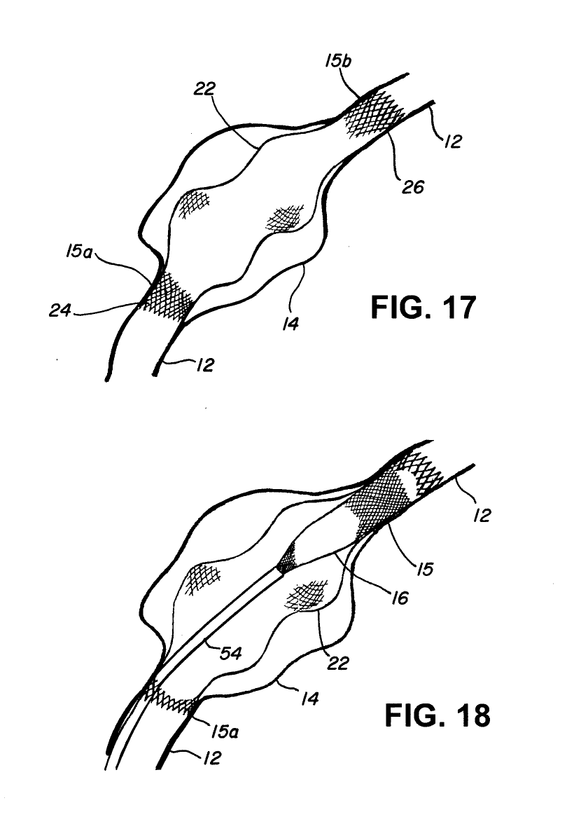

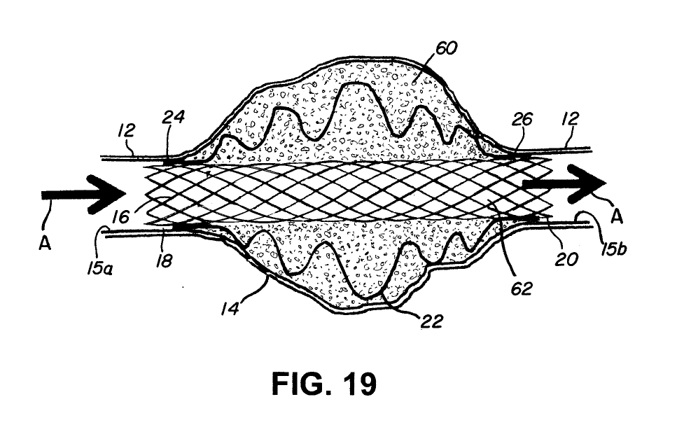

In some embodiments, the inner flow disruption element 16 and/or the outer flow disruption element 22 may be constructed with two or more sizes of filaments, as shown in FIGS. 11 and 12. For example, an inner flow disruption element 16′ (FIG. 11) may be made of a multitude of pore-defining filaments 32 of a first diameter, and several support filaments 33 having a second diameter greater than the first diameter. The larger-diameter support filaments 33 provide structural support and shape definition for the flow disruption element, while the smaller-diameter pore-defining filaments 32 define an arrangement of pores 34 that provides inner element wall with a porosity (a function of pore size and pore density) that provides the desired flow resistance to reduce blood flow advantageously to the thrombogenic threshold velocity (as defined below). For example, the pore-defining filaments 32 may have a transverse dimension or diameter of about 0.015 mm to about 0.05 mm for some embodiments, and about 0.01 mm to about 0.025 mm in other embodiments. The support filaments 33 may have a transverse dimension or diameter of about 0.04 mm to about 0.1 mm in some embodiments, and about 0.025 mm to about 0.1 mm in other embodiments. The ratio of small filaments 32 to large filaments 33 is advantageously greater than about 3 to 1, such as, for example, 4 to 1 and 10 to 1. The filaments 30, 32 may be braided in a plain weave that is one under, one over structure or a supplementary weave; more than one warp interlace with one or more than one weft. Braid wire density is described as picks per inch (PPI), which is the number of wire crossovers per inch. The PPI or pick count of a braided element may be varied between about 50 and 300 picks per inch (PPI). In some embodiments, the PPI of the inner flow disruption element 16 may be about 2-20 times the PPI of the outer flow disruption element 22.

Any of the device embodiments and components described herein may include metals, polymers, biologic materials and composites thereof. Suitable metals include zirconium-based alloys, cobalt-chrome alloys, nickel-titanium alloys, platinum, tantalum, stainless steel, titanium, gold, and tungsten. Potentially suitable polymers include, but are not limited to, acrylics, silk, silicones, polyvinyl alcohol, polypropylene, polyesters (e.g. polyethylene terephthalate or PET), PolyEtherEther Ketone (PEEK), polytetrafluoroethylene (PTFE), polycarbonate urethane (PCU), polyurethane (PU), and high molecular weight polyethylene. Device embodiments may include a material that degrades or is absorbed or eroded by the body. A bioresorbable (e.g., breaks down and is absorbed by a cell, tissue, or other mechanism within the body) or bioabsorbable (similar to bioresorbable) material may be used. Alternatively, a bioerodable (e.g., erodes or degrades over time by contact with surrounding tissue fluids, through cellular activity or other physiological degradation mechanisms), or biodegradable (e.g., degrades over time by enzymatic or hydrolytic action, or other mechanism in the body) polymer or dissolvable material may be employed. Each of these terms is interpreted to be interchangeable. Potentially suitable bioabsorbable materials include polylactic acid (PLA), poly(alpha-hydroxy) acids, such as poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen, poly(hydroxybutyrate), polyanhydride, polyphosphoester, poly(amino acids), or related copolymer materials. An absorbable composite fiber may be made by combining a reinforcement fiber made from a copolymer of about 18% glycolic acid and about 82% lactic acid with a matrix material consisting of a blend of the above copolymer with about 20% polycaprolactone (PCL).

For some embodiments, the pore defining filaments 32 define pores or openings 34 that may have an elongated, substantially diamond shape, as best shown, for example, in FIG. 12. The diamond shaped pores or openings 34 may have a width substantially less than the length to provide greater radial strength. In some embodiments, the ratio of diamond shaped pore opening length to width may exceed a ratio of 3 to 1. The pore size is defined by the largest circular shapes that may be disposed within the pores or openings 34 without displacing or distorting the filaments that define each of the openings or pores 34. For example, in many embodiments, the pore size may range from about 0.13 mm to about 0.25 mm, more specifically, about 0.15 mm to about 0.23 mm, and even more specifically, about 0.18 mm to about 0.20 mm. In other embodiments, the pore size may be as large as about 0.3 to 0.4 mm, or even as large as about 1.0 mm. The inner and/or outer flow disruption elements may be constructed either with a substantially uniform pore size, or with two or more different pore sizes.

In some embodiments, there are openings in the inner element 16 and the outer element, wherein the largest of the openings is configured to allow blood flow through the openings at a velocity below a thrombotic threshold velocity. Thus, blood flow within the aneurysm may be substantially slowed to below the thrombogenic threshold velocity. Thrombogenic threshold velocity has been defined as the time-average velocity at which more than 50% of a vascular graft surface is covered by thrombus when deployed within a patient's vasculature. In the context of aneurysm occlusion, a slightly different definition of the thrombogenic threshold velocity may be appropriate. Accordingly, the term “thrombotic threshold velocity” as used herein shall include the velocity at which clotting occurs within or on a device, such as the device 10 described herein, deployed within a patient's vasculature, such that blood flow into a vascular defect treated by the device is substantially blocked in less than about 1 hour or otherwise during the treatment procedure. The blockage of blood flow into the vascular defect may be indicated in some cases by minimal contrast agent entering the vascular defect after a sufficient amount of contrast agent has been injected into the patient's vasculature upstream of the implant site and visualized as it dissipates from that site. Thus, in some embodiments, substantially no contrast agent will be seen on a post treatment angiogram in less than about 1 hour. Such sustained diversion of flow within less than about 1 hour or during the duration of the implantation procedure may also be referred to as acute stasis or occlusion of the vascular defect.

As noted above, the flow disruption elements 16, 22 may be formed at least in part of wire, ribbon, or other filamentary members. These filamentary members may have circular, elliptical, ovoid, square, rectangular, or triangular cross-sections. The flow disruption elements 16, 22 may also be formed using conventional machining, laser cutting, electrical discharge machining (EDM) or photochemical machining (PCM). If made of a metal, they may be formed from either metallic tubes or sheet material.

For braided portions, components, or elements, the braiding process may be carried out by automated machine fabrication or may also be performed by hand. For some embodiments, the braiding process may preferentially be carried out by the braiding apparatus and process described in commonly assigned U.S. patent application Ser. No. 13/275,264, now U.S. Pat. No. 8,261,648, Braiding Mechanism and Methods of Use by Marchand et al., which is herein incorporated in its entirety by reference. As shown, for example, in FIG. 13, a plurality of elongate resilient filaments 36 is secured at one end of an elongate cylindrical braiding mandrel 38 by a constraining band 40. The band 40 may include any suitable structure that secures the ends of the filaments 36 relative to the mandrel 38, such as a band of adhesive tape, an elastic band, an annular clamp or the like. The loose ends of the filaments opposite the secured ends are manipulated into a braided or woven pattern to achieve the braid pattern for generation of a braided tubular member.

In some embodiments, a braiding mechanism may be utilized that comprises a disc defining a plane and a circumferential edge, a mandrel extending from a center of the disc and generally perpendicular to the plane of the disc, and a plurality of actuators positioned circumferentially around the edge of the disc. A plurality of filaments is loaded on the mandrel such that each filament extends radially toward the circumferential edge of the disc and each filament contacts the disc at a point of engagement on the circumferential edge, which is spaced apart a discrete distance from adjacent points of engagement. The point at which each filament engages the circumferential edge of the disc is separated by a distance d from the points at which each immediately adjacent filament engages the circumferential edge of the disc. The disc and a plurality of catch mechanisms are configured to move relative to one another to rotate a first subset of filaments relative to a second subset of filaments to interweave the filaments. The first subset of the plurality of filaments is engaged by the actuators, and the plurality of actuators is operated to move the engaged filaments in a generally radial direction to a position beyond the circumferential edge of the disc. The disc is then rotated a first direction by a circumferential distance, thereby rotating a second subset of filaments a discrete distance and crossing the filaments of the first subset over the filaments of the second subset. The actuators are operated again to move the first subset of filaments to a radial position on the circumferential edge of the disc, wherein each filament in the first subset is released to engage the circumferential edge of the disc at a circumferential distance from its previous point of engagement.

In some embodiments, the braiding apparatus provides for a disc that is rotated by a circumferential distance, and the plurality of catch mechanisms is then operated to engage every other filament and pull the engaged filaments in a generally radial direction to a position beyond the circumferential edge of the disc. The point at which each filament engages the circumferential edge of the disc is separated by a distance d from the points at which each immediately adjacent filament engages the circumferential edge of the disc. The disc is then rotated in a second, opposite direction by a circumferential distance; and the plurality of catch mechanisms is operated to release each engaged filament radially toward the circumferential edge of the disc, wherein each filament is placed in an empty notch located a circumferential distance from the notch formerly occupied. In some embodiments, the disc is rotated by a circumferential distance 2d in the first direction. In some embodiments, the disc may further be rotated by a circumferential distance 2d in the second direction.

As discussed above, although a one over-one under simple braid pattern is shown and discussed, other braid or weave patterns may also be used. One such example of another braid configuration may include a two over-one under pattern. Once the braided tubular member achieves sufficient length, it may be removed from the braiding mandrel 38 and positioned within a shaping fixture (not shown) for further shape setting. In some embodiments, the filamentary elements of a flow disruption element may be held by a fixture configured to hold the porous element in a desired shape and heated to about 475-525 degrees Celsius for about 5-15 minutes to shape-set the structure.

For embodiments where the filaments are metal wire, the characteristics of the filament materials may be altered by heat treating the wire. By locally treating portions of a metal wire, it is possible to produce a metal wire with spatial variations in the elasticity and stiffness of the metal. The locally treated portions will initiate plastic deformation at a lower strain than the portions that have not been locally treated. The localized heat treatment of the desired metal wire filaments may be accomplished by any suitable method. One such suitable method involves the use of electrical resistance heating. Electrical leads are attached across the desired portion of the element, and a current is passed through it. Because of the resistance of the shape-memory metal, the desired portion of metal heats up, thereby further annealing the material. Another suitable method for local heat treatment involves applying a heated inert gas jet to a desired portion of the element to selectively heat a desired portion of the element. Yet another method involves the use of an induction coil that is placed over a desired portion of the element to effect induction heating of the desired portion. A laser may also be used to selectively heat desired regions of the element. The desired regions of the element may also be brazed. The element may also be placed in a heat-treating fluid, such as a salt bath or a fluidized sand bath, with appropriate sections of the element insulated.

In any of the embodiments described, the inner and/or outer flow disruption elements may comprise a material with low bioactivity and good hemocompatibility, so as to minimize platelet aggregation or attachment and thus the propensity to form clots and thrombi. Optionally, the inner element 16 may be coated, or it may incorporate an antithrombogenic agent such as heparin or other antithrombogenic agents described herein or known in the art. Antiplatelet agents may include aspirin, glycoprotein IIb/IIIa receptor inhibitors (including, abciximab, eptifibatide, tirofiban, lamifiban, fradafiban, cromafiban, toxifiban, XV454, lefradafiban, klerval, lotrafiban, orbofiban, and xemilofiban), dipyridamole, apo-dipyridamole, persantine, prostacyclin, ticlopidine, clopidogrel, cromafiban, cilostazol, and nitric oxide. To deliver nitric oxide, device embodiments may include a polymer that releases nitric oxide. Device embodiments may also deliver or include an anticoagulant such as heparin, low molecular weight heparin, hirudin, warfarin, bivalirudin, hirudin, argatroban, forskolin, ximelagatran, vapiprost, prostacyclin and prostacyclin analogues, dextran, synthetic antithrombin, Vasoflux, argatroban, efegatran, tick anticoagulant peptide, Ppack, HMG-CoA reductase inhibitors, and thromboxane A2 receptor inhibitors.

The outer flow disruption element(s) may comprise materials with high bioactivity and/or high thrombogenicity and thus enhance the formation of an occlusive mass of clot within the vascular defect and thus embolization. Some materials that have been shown to have high bioactivity and/or high thrombogenicity include silk, polylactic acid (PLA), polyglycolic acid (PGA), collagen, alginate, fibrin, fibrinogen, fibronectin, methylcellulose, gelatin, small Intestinal submucosa (SIS), poly-N-acetylglucosamine and copolymers or composites thereof.

Bioactive agents suitable for use in the embodiments discussed herein may include those having a specific action within the body as well as those having nonspecific actions. Specific action agents are typically proteinaceous, including thrombogenic types and/or forms of collagen, thrombin and fibrogen (each of which may provide an optimal combination of activity and cost), as well as elastin and von Willebrand factor (which may tend to be less active and/or expensive agents), and active portions and domains of each of these agents. Thrombogenic proteins typically act by means of a specific interaction with either platelets or enzymes that participate in a cascade of events leading eventually to clot formation. Agents having nonspecific thrombogenic action are generally positively charged molecules, e.g., polymeric molecules such as chitosan, polylysine, poly(ethylenimine) or acrylics polymerized from acrylimide or methacrylamide which incorporate positively-charged groups in the form of primary, secondary, or tertiary amines or quarternary salts, or non-polymeric agents such as (tridodecylmethylammonium chloride). Positively charged hemostatic agents promote clot formation by a non-specific mechanism, which includes the physical adsorption of platelets via ionic interactions between the negative charges on the surfaces of the platelets and the positive charges of the agents themselves.

Embodiments described herein may include a surface treatment or coating on at least some surfaces that promotes or inhibits thrombosis, clotting, healing or other embolization performance measure. The surface treatment or coating may be a synthetic, biologic or combination thereof. For some embodiments, at least a portion of the device may have a surface treatment or coating made of a biodegradable or bioresorbable material such as a polylactide, polyglycolide or a copolymer thereof. Another surface treatment or coating material which may enhance the embolization performance of a device includes a polysaccharide such as an alginate based material. Some coating embodiments may include extracellular matrix proteins such as ECM proteins. One example of such a coating may be Finale Prohealing coating which is commercially available from Surmodics Inc., Eden Prairie, Minn. Another exemplary coating may be Polyzene-F which is commercially available from CeloNovo BioSciences, Inc., Newnan, Ga. In some embodiments, the coatings may be applied with a thickness that is less than about 25% of a transverse dimension of the filaments. In some embodiments, at least a portion of at least one of the flow disruption elements 16, 22 may be coated with a composition that may include nanoscale structured materials or precursors thereof (e.g., self-assembling peptides). The peptides may have alternating hydrophilic and hydrophobic monomers that allow them to self-assemble under physiological conditions.

Device embodiments discussed herein may be delivered and deployed from a delivery and positioning system 50 (FIG. 14) that includes an access sheath 52 and a microcatheter 54 (FIGS. 15 and 17), such as the type of microcatheter that is known in the art of neurovascular navigation and therapy. Device embodiments for treatment of a patient's vasculature may be elastically collapsed and restrained by a tube or other radial restraint, such as an inner lumen of the microcatheter 54, for delivery and deployment, as will be described below. As shown in FIG. 14, the access sheath 52 with the microcatheter 54 may generally be inserted through a small incision accessing a peripheral blood vessel such as the femoral artery 56 or the radial artery 58. The microcatheter 54 may be delivered or otherwise navigated to a desired treatment site 60 from a position outside the patient's body over a guidewire (not shown) under fluoroscopy or by other suitable guiding methods, as are well-known in the art. The guidewire may be removed during such a procedure to allow insertion of the device 10 secured to a delivery apparatus of the delivery system 50 through the inner lumen of the microcatheter 54 in some cases.

Access to a variety of blood vessels of a patient may be established, including arteries such as the femoral artery, radial artery, and the like in order to achieve percutaneous access to a vascular defect. In general, the patient may be prepared for surgery, the access artery is exposed via a small surgical incision, and access to the lumen is gained using the Seldinger technique where an introducing needle is used to place a wire over which a dilator or series of dilators dilates a vessel allowing an introducer sheath to be inserted into the vessel. This would allow the device to be used percutaneously. With an introducer sheath in place, a guiding catheter is then used to provide a safe passageway from the entry site to a region near the target site to be treated. For example, in treating a site in the human brain, a guiding catheter (not shown) would be chosen that would extend from the entry site at the femoral artery up through the large arteries extending around the heart through the aortic arch, and downstream through one of the arteries extending from the upper side of the aorta, such as the carotid artery. Typically, a guidewire and neurovascular microcatheter 54 are then placed through the guiding catheter and advanced through the patient's vasculature, until a distal end of the microcatheter 54 is disposed adjacent the target vascular defect, such as an aneurysm. Exemplary guidewires for neurovascular use include the Synchro2® made by Boston Scientific and the Glidewire Gold Neuro® made by MicroVention Terumo. Typical guidewire sizes may include 0.014 inches (0.36 mm) and 0.018 inches (0.46 mm). Once the distal end of the microcatheter 54 is positioned at the site, often by locating its distal end through the use of radiopaque marker material and fluoroscopy, the microcatheter 54 is cleared. For example, if a guidewire has been used to position the microcatheter 54, the guidewire is withdrawn from the microcatheter 54, and then the implant delivery apparatus is advanced through the microcatheter 54.

The device 10 may be releasably secured to the distal end of a delivery apparatus, as is known in the art of endovascular stent delivery. An exemplary delivery system is described in U.S. Patent Application 2008/0288043, the disclosure of which is herein incorporated by reference in its entirety.

For delivery and deployment, the above-described blood flow disruption device 10 is first compressed to a radially constrained and longitudinally flexible state, then installed into the proximal end of a microcatheter 54. The microcatheter is then introduced intravascularly, as described above, until its distal end is positioned for deployment of the device 10, as described below. After deployment of the device 10, the microcatheter 54 is withdrawn.

More specifically, as shown in FIG. 15, when the distal end of the microcatheter 54 is located for deployment of the device 10, the device 10 is advanced distally through the lumen 55 of the microcatheter 54 by a delivery mechanism. The delivery mechanism, in the illustrated embodiment, comprises a flexible pusher wire 56 and, advantageously, a flexible engagement sleeve 58 that maintains an engagement between the pusher wire 56 and the blood flow disruption device 10 while the device is advanced through the microcatheter 54. In one embodiment, the engagement sleeve 58 is a hollow flexible tube having an outer diameter that is slightly smaller than the inner diameter of the microcatheter 54, and with an inner diameter that is large enough to contain the blood flow disruption device 10 in the latter's collapsed state. The pusher wire 56 is sized to fit within the lumen of the hollow engagement sleeve 58. With the device 10 installed in its distal end, the engagement sleeve 58 is advanced though the lumen of the microcatheter 54 until the device 10 is located proximate the distal end of the microcatheter. The pusher wire 56 is then advanced distally within the engagement sleeve 58 so as to push the device 10 out of the distal end of the microcatheter 54 for deployment, after which the engagement sleeve 58 is withdrawn proximally through the microcatheter. Thus, the device is released by the relative movement between the engagement sleeve 58 and the microcatheter, allowing the operating surgeon to manipulate the microcatheter and thereby change the position of the device in situ.

Other delivery mechanisms that may be adapted to deploy the flow disruption device of the present disclosure are known in the art. See, for example, US 2009/0318947 and U.S. Pat. No. 6,425,898, the disclosures of which are incorporated herein in their entirety.

In other embodiments, the microcatheter 54 may first be navigated to a desired treatment site over a guidewire (not shown) or by other suitable navigation techniques. The distal end of the microcatheter 54 may be positioned such that it is directed towards or disposed adjacent the vascular defect to be treated, and the guidewire is then withdrawn. The device 10, secured to a suitable delivery mechanism (such as that described above), may then be radially constrained, inserted into a proximal portion of the inner lumen of the microcatheter 54, and distally advanced to the vascular defect through the lumen of the microcatheter 54.

In some embodiments, the device 10 is made as a unitary structure with the inner and outer flow disruption elements 16, 22 attached to each other and thus deployed together into the vessel in accordance with one of the deployment methods described above. In other embodiments, the inner and outer flow disruption elements 16, 22 are deployed separately. A method of deploying the flow disruption elements 16, 22 of the device 10 sequentially in a blood vessel 12 having a vascular wall defect 14 in the form of a fusiform aneurysm, as discussed above with reference to FIG. 1, is illustrated in FIGS. 16-18. As shown in FIG. 16, the distal end of a microcatheter 54, which has been loaded with an outer flow disruption element 22 in a compressed or collapsed state, is guided to a target vascular defect 14 in the manner described above, and the outer flow disruption element 22 is then ejected from the distal end of the microcatheter 54 into blood vessel 12 distally from the aneurysm 14. This results in the seating of outflow end 26 of the outer flow disruption element 22 in the downstream vessel portion 15b.

The ejection of the outer flow disruption element 22 continues, as the microcatheter 54 is withdrawn proximally through the aneurysm 14, so that the outer flow disruption element 22 bridges the aneurysm 14. When the microcatheter has been fully withdrawn, the result is an outer flow disruption element 22 that expands radially into its expanded state within the aneurysm 14, and that has its outflow end 26 seated in the distal or downstream vascular segment 15b (as mentioned above), and its inflow end 24 seated in the proximal or upstream vascular segment 15a, as shown in FIG. 17.

Next, an inner flow disruption element 16 is loaded, in a collapsed state, into a microcatheter 54, and the microcatheter is guided to the target vascular site once again. The inner flow disruption element 16 is then ejected from the distal end of the microcatheter 54 into the blood vessel 12 distally from the aneurysm 14, seating the distal fixation zone 20 in the distal or downstream vascular 15b, as shown in FIG. 18. Finally, the microcatheter 54 is again withdrawn proximally through the aneurysm so that the inner flow disruption element 16 bridges or spans the aneurysm 14. When the microcatheter is fully withdrawn, the inner flow disruption element 16 expands radially into its expanded state within the aneurysm 14, with its distal fixation zone 20 seated in the distal or downstream vascular segment 15b, and its proximal fixation zone 18 seated in the proximal or upstream vascular segment 15a, as shown in FIG. 1. The installation of the inner flow disruption element 16 in this manner captures the outflow end 26 of the outer flow disruption element 22 between the distal fixation zone 20 of the inner flow disruption element 16 and the distal or downstream vascular segment 15b, while inflow end 24 of the outer flow disruption element 22 is captured between the proximal fixation zone 18 of the inner flow disruption element 16 and the proximal or upstream vascular segment 15a.

FIG. 19 shows the device 10 installed in an aneurysm 14 after the aneurysm has been filled by an embolism 60 formed by means of the hemostasis promoted by the device, as discussed above. The inner flow disruption element 16 forms a fixed stent that provides a reconstructed or reconstituted blood flow passage or lumen 62 through the embolism 60 from the upstream vascular segment 15a to the downstream vascular segment 15b. The outer flow disruption element 22 forms a web or matrix that supports the embolism 60 and fixes it in place in the aneurysm 14.

While several embodiments have been described herein, it is understood that these embodiments are exemplary only, and that other embodiments, variations, and modifications will suggest themselves to those skilled in the pertinent arts. Such other embodiments, variations and modifications are considered to be within the spirit and scope of the present disclosure.

What is claimed is:

1. A blood flow disruption device for embolizing an interior portion of an aneurysm, the device comprising:
a first self-expanding braided element having a radially collapsed state for delivery through a catheter and a radially expanded state having an undulating form, the first self-expanding braided element comprising a first plurality of nitinol filaments; and
a second self-expanding braided element adjacent the first self-expanding element and having a radially collapsed state for delivery through a catheter and a radially expanded state having an undulating form, the second self-expanding braided element comprising a second plurality of nitinol filaments;
wherein the first self-expanding braided element and the second self-expanding braided element are configured to be placed within an aneurysm such that in their radially expanded states at least a portion of the first self-expanding braided element is disposed within a cavity formed by the second self-expanding braided element.

2. The device of claim 1, wherein the first self-expanding braided element and the second self-expanding braided element are attached to each other by at least one attachment method selected from the group consisting of welding, brazing, soldering, and adhesive bonding.

3. The device of claim 1, wherein at least one of the first plurality of nitinol filaments and the second plurality of nitinol filaments comprises super-elastic nickel-titanium alloy.

4. The device of claim 1, wherein the first self-expanding braided element and the second self-expanding braided element are configured to be placed within a fusiform aneurysm.

5. The device of claim 1, wherein the first self-expanding braided element and the second self-expanding braided element are configured to be placed within a wide neck aneurysm.

6. The device of claim 1, wherein at least one of the first plurality of nitinol filaments and second plurality of nitinol filaments comprises filaments each having a transverse dimension or diameter of about 0.015 mm to about 0.05 mm.

7. The device of claim 1, wherein at least one of the first plurality of nitinol filaments and second plurality of nitinol filaments comprises filaments each having a transverse dimension or diameter of about 0.01 mm to about 0.025 mm.

8. The device of claim 1, wherein at least one of the first plurality of nitinol filaments and second plurality of nitinol filaments comprises larger filaments and smaller filaments, the larger filaments each having a transverse dimension or diameter that is greater than the transverse dimension or diameter of the smaller filaments.

9. The device of claim 8, wherein each of the larger filaments has a transverse dimension or diameter of about 0.015 mm to about 0.05 mm, and each of the smaller filaments has a transverse dimension or diameter of about 0.01 mm to about 0.025 mm.

10. The device of claim 8, wherein the ratio of the number of smaller filaments to the number of larger filaments is greater than about 3 to 1.

11. The device of claim 8, wherein the ratio of the number of smaller filaments to the number of larger filaments is between about 4 to 1 and 10 to 1.

12. The device of claim 1, wherein at least one of the first self-expanding braided element and the second self-expanding braided element has a braid wire density of between about 50 and 300 picks per inch.

13. The device of claim 1, wherein at least one of the first self-expanding braided element and the second self-expanding braided element has a pore size of between about 0.13 mm and about 0.25 mm.

14. The device of claim 1, wherein at least one of the first self-expanding braided element and the second self-expanding braided element has a pore size of between about 0.15 mm and about 0.23 mm.

15. The device of claim 1, wherein at least one of the first self-expanding braided element and the second self-expanding braided element has a pore size of between about 0.18 mm and about 0.20 mm.

16. The device of claim 1, wherein at least one of the first self-expanding braided element and the second self-expanding braided element includes helical concavities.

17. The device of claim 1, wherein at least one of the first self-expanding braided element and the second self-expanding braided element comprises a one under, one over structure.

18. The device of claim 1, wherein at least one of the first plurality of nitinol filaments and second plurality of nitinol filaments comprises filaments each having a circular cross-section.

19. The device of claim 1, further comprising micro-mechanical means for removable attachment of the device to a delivery apparatus.
